(19) United States
(12) Patent Application Publication
(10) Pub. No.: US 2025/0188016 A1
(43) Pub. Date: Jun. 12, 2025

(54) CATIONIC LIPID COMPOUNDS FOR USE IN LIPID NANOPARTICLES

(71) Applicant: Acuitas Therapeutics, Inc., Vancouver (CA)

(72) Inventor: Steven M. Ansell, Vancouver (CA)

(21) Appl. No.: 18/977,214

(22) Filed: Dec. 11, 2024

Related U.S. Application Data
(60) Provisional application No. 63/609,107, filed on Dec. 12, 2023.

(51) Int. Cl. C07C 219/06; A61K 9/51; A61K 45/06

(52) U.S. Cl. CPC C07C 219/06; A61K 9/5123; A61K 45/06

(57) ABSTRACT

Compounds are provided having the following Formula (I):

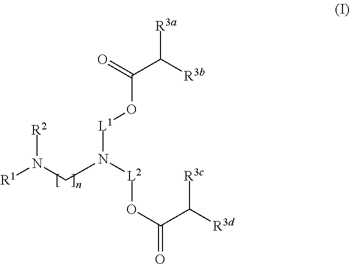

or a pharmaceutically acceptable salt, tautomer, or stereoisomer thereof, wherein R¹, R², R^{3a}, R^{3b}, R^{3c}, R^{3d}, L¹, L², and n are as defined herein. Use of the compounds as a component of lipid nanoparticle formulations for delivery of a therapeutic agent, compositions comprising the compounds and methods for their use and preparation are also provided.

### BACKGROUND

### Technical Field

The present disclosure generally relates to novel cationic lipids that can be used in combination with other lipid components, such as neutral lipids, cholesterol, and polymer conjugated lipids, to form lipid nanoparticles with oligonucleotides, to facilitate the intracellular delivery of therapeutic nucleic acids (e.g., oligonucleotides, messenger RNA) both in vitro and in vivo.

### Description of the Related Art

There are many challenges associated with the delivery of nucleic acids to affect a desired response in a biological system. Nucleic acid-based therapeutics have enormous potential but there remains a need for more effective delivery of nucleic acids to appropriate sites within a cell or organism to realize this potential. Therapeutic nucleic acids include, e.g., messenger RNA (mRNA), antisense oligonucleotides, ribozymes, DNAzymes, plasmids, immune stimulating nucleic acids, antagomir, antimir, mimic, supermir, and aptamers. Some nucleic acids, such as mRNA or plasmids, can be used to effect expression of specific cellular products as would be useful in the treatment of, for example, diseases related to a deficiency of a protein or enzyme. The therapeutic applications of translatable nucleotide delivery are extremely broad as constructs can be synthesized to produce any chosen protein sequence, whether indigenous to the system. The expression products of the nucleic acid can augment existing levels of protein, replace missing or non-functional versions of a protein, or introduce a new protein and associated functionality in a cell or organism.

Some nucleic acids, such as miRNA inhibitors, can be used to effect expression of specific cellular products that are regulated by miRNA as would be useful in the treatment of, for example, diseases related to deficiency of protein or enzyme. The therapeutic applications of miRNA inhibition are extremely broad as constructs can be synthesized to inhibit one or more miRNA that would in turn regulate the expression of mRNA products. The inhibition of endogenous miRNA can augment its downstream target endogenous protein expression and restore proper function in a cell or organism as a means to treat disease associated to a specific miRNA or a group of miRNA.

Other nucleic acids can down-regulate intracellular levels of specific mRNA and, as a result, down-regulate the synthesis of the corresponding proteins through processes such as RNA interference (RNAi) or complementary binding of antisense RNA. The therapeutic applications of antisense oligonucleotide and RNAi are also extremely broad, since oligonucleotide constructs can be synthesized with any nucleotide sequence directed against a target mRNA. Targets may include mRNAs from normal cells, mRNAs associated with disease-states, such as cancer, and mRNAs of infectious agents, such as viruses. To date, antisense oligonucleotide constructs have shown the ability to specifically down-regulate target proteins through degradation of the cognate mRNA in both in vitro and in vivo models. In addition, antisense oligonucleotide constructs are currently being evaluated in clinical studies.

However, two problems currently face using oligonucleotides in therapeutic contexts. First, free RNAs are susceptible to nuclease digestion in plasma. Second, free RNAs have limited ability to gain access to the intracellular compartment where the relevant translation machinery resides. Lipid nanoparticles formed from lipid components, such as cationic lipids, neutral lipids, cholesterol, PEG, PEGylated lipids, and oligonucleotides have been used to block degradation of the RNAs in plasma and facilitate the cellular uptake of the oligonucleotides.

There remains a need for improved cationic lipids and lipid nanoparticles for the delivery of oligonucleotides. Preferably, these lipid nanoparticles would provide optimal drug to lipid ratios, protect the nucleic acid from degradation and clearance in serum, be suitable for systemic delivery, and provide intracellular delivery of the nucleic acid. In addition, these lipid-nucleic acid particles should be well-tolerated and provide an adequate therapeutic index, such that patient treatment at an effective dose of the nucleic acid is not associated with unacceptable toxicity and/or risk to the patient. The present disclosure provides these and related advantages.

### BRIEF SUMMARY

In brief, the present disclosure provides lipid compounds, including stereoisomers, pharmaceutically acceptable salts, or tautomers thereof, which can be used alone or in combination with other lipid components such as neutral lipids, charged lipids, steroids (including for example, all sterols) and/or their analogs, and/or polymer conjugated lipids to form lipid nanoparticles for the delivery of therapeutic agents. In some instances, the lipid nanoparticles are used to deliver nucleic acids such as antisense and/or messenger RNA. Methods for use of such lipid nanoparticles for treatment of various diseases or conditions, such as those caused by infectious entities and/or insufficiency of a protein, are also provided.

In one embodiment, compounds having the following Formula (I) are provided:

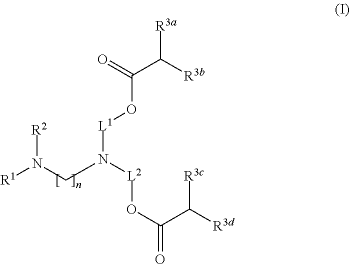

or a pharmaceutically acceptable salt, tautomer, or stereoisomer thereof, wherein R¹, R², R^{3a}, R^{3b}, R^{3c}, R^{3d}, L¹, L², and n are as defined herein.

Pharmaceutical compositions comprising one or more of the foregoing compounds of Formula (I) and a therapeutic agent are also provided. In some embodiments, the pharmaceutical compositions further comprise one or more components selected from neutral lipids, charged lipids, steroids, and polymer conjugated lipids. Such compositions are useful for formation of lipid nanoparticles for the delivery of the therapeutic agent.

In other embodiments, the present disclosure provides a method for administering a therapeutic agent to a patient in need thereof, the method comprising preparing a composition of lipid nanoparticles comprising the compound of Formula (I) and a therapeutic agent and delivering the composition to the patient. Such methods are useful for inducing expression of a protein in a subject, for example for expressing an antigen for purposes of vaccination or a gene editing protein.

These and other aspects of the disclosure will be apparent upon reference to the following detailed description.

### DETAILED DESCRIPTION

In the following description, certain specific details are set forth to provide a thorough understanding of various embodiments of the disclosure. However, one skilled in the art will understand that the embodiments of the disclosure may be practiced without these details.

The present disclosure is based, in part, upon the discovery of novel cationic lipids that provide advantages when used in lipid nanoparticles for the in vivo delivery of an active or therapeutic agent such as a nucleic acid into a cell of a mammal. In some embodiments of the present disclosure provide nucleic acid-lipid nanoparticle compositions comprising one or more of the novel cationic lipids described herein that provide increased activity of the nucleic acid and improved tolerability of the compositions in vivo, resulting in a significant increase in the therapeutic index as compared to nucleic acid-lipid nanoparticle compositions previously described.

In some embodiments, the present disclosure provides novel cationic that enable the formulation of improved compositions for the in vitro and in vivo delivery of mRNA and/or other oligonucleotides. In some embodiments, these improved lipid nanoparticle compositions are useful for expression of protein encoded by mRNA. In other embodiments, these improved lipid nanoparticles compositions are useful for upregulation of endogenous protein expression by delivering miRNA inhibitors targeting one specific miRNA or a group of miRNA regulating one target mRNA or several mRNA. In other embodiments, these improved lipid nanoparticle compositions are useful for down-regulating (e.g., silencing) the protein levels and/or mRNA levels of target genes. In some other embodiments, the lipid nanoparticles are also useful for delivery of mRNA and plasmids for expression of transgenes. In yet other embodiments, the lipid nanoparticle compositions are useful for inducing a pharmacological effect resulting from expression of a protein, e.g., increased production of red blood cells through the delivery of a suitable erythropoietin mRNA, or protection against infection through delivery of mRNA encoding for a suitable antibody.

The lipid nanoparticles and compositions of the present disclosure may be used for a variety of purposes, including the delivery of encapsulated or associated (e.g., complexed) therapeutic agents such as nucleic acids to cells, both in vitro and in vivo. Accordingly, embodiments of the present disclosure provide methods of treating or preventing diseases or disorders in a subject in need thereof by contacting the subject with a lipid nanoparticle that encapsulates or is associated with a suitable therapeutic agent, wherein the lipid nanoparticle comprises one or more of the novel cationic lipids described herein.

As described herein, embodiments of the lipid nanoparticles of the present disclosure are particularly useful for the delivery of nucleic acids, including, e.g., mRNA, antisense oligonucleotide, plasmid DNA, microRNA (miRNA), miRNA inhibitors (antagomirs/antimirs), messenger-RNA-interfering complementary RNA (micRNA), DNA, multivalent RNA, dicer substrate RNA, complementary DNA (cDNA), etc. Therefore, the lipid nanoparticles and compositions of the present disclosure may be used to induce expression of a desired protein both in vitro and in vivo by contacting cells with a lipid nanoparticle comprising one or more novel cationic lipids described herein, wherein the lipid nanoparticle encapsulates or is associated with a nucleic acid that is expressed to produce the desired protein (e.g., a messenger RNA or plasmid encoding the desired protein). Alternatively, the lipid nanoparticles and compositions of the present disclosure may be used to decrease the expression of target genes and proteins both in vitro and in vivo by contacting cells with a lipid nanoparticle comprising one or more novel cationic lipids described herein, wherein the lipid nanoparticle encapsulates or is associated with a nucleic acid that reduces target gene expression (e.g., an antisense oligonucleotide or small interfering RNA (siRNA)). The lipid nanoparticles and compositions of the present disclosure may also be used for co-delivery of different nucleic acids (e.g., mRNA and plasmid DNA) separately or in combination, such as may be useful to provide an effect requiring colocalization of different nucleic acids (e.g., mRNA encoding for a suitable gene modifying enzyme and DNA segment(s) for incorporation into the host genome).

Nucleic acids for use with this disclosure may be prepared according to any available technique. For mRNA, the primary methodology of preparation is, but not limited to, enzymatic synthesis (also termed in vitro transcription) which currently represents the most efficient method to produce long sequence-specific mRNA. In vitro transcription describes a process of template-directed synthesis of RNA molecules from an engineered DNA template comprised of an upstream bacteriophage promoter sequence (e.g., including but not limited to that from the T7, T3, and SP6 coliphage) linked to a downstream sequence encoding the gene of interest. Template DNA can be prepared for in vitro transcription from a number of sources with appropriate techniques which are well known in the art including, but not limited to, plasmid DNA and polymerase chain reaction amplification (see Linpinsel, J. L. and Conn, G. L., General protocols for preparation of plasmid DNA template and Bowman, J. C., Azizi, B., Lenz, T. K., Ray, P., and Williams, L. D. in RNA in vitro transcription and RNA purification by denaturing PAGE in Recombinant and in vitro RNA syntheses Methods v. 941 Conn G. L. (ed), New York, N.Y. Humana Press, 2012).

Transcription of the RNA occurs in vitro using the linearized DNA template in the presence of the corresponding RNA polymerase and adenosine, guanosine, uridine, and cytidine ribonucleoside triphosphates (rNTPs) under conditions that support polymerase activity while minimizing potential degradation of the resultant mRNA transcripts. In vitro transcription can be performed using a variety of commercially available kits including, but not limited to RiboMax Large Scale RNA Production System (Promega), MegaScript Transcription kits (Life Technologies) as well as with commercially available reagents including RNA polymerases and rNTPs. The methodology for in vitro transcription of mRNA is well known in the art. (see, e.g., Losick, R., 1972, In vitro transcription, Ann Rev Biochem v.41 409-46; Kamakaka, R. T. and Kraus, W. L. 2001. In Vitro Transcription. Current Protocols in Cell Biology. 2:11.6:11.6.1-11.6.17; Beckert, B. And Masquida, B., (2010) Synthesis of RNA by In Vitro Transcription in RNA in Methods in Molecular Biology v. 703 (Neilson, H. Ed), New York, N.Y. Humana Press, 2010; Brunelle, J. L. and Green, R., 2013, Chapter Five—In vitro transcription from plasmid or PCR-amplified DNA, Methods in Enzymology v. 530, 101-114; all of which are incorporated herein by reference).

The desired in vitro transcribed mRNA is then purified from the undesired components of the transcription or associated reactions (including unincorporated rNTPs, protein enzyme, salts, short RNA oligos etc.). Techniques for the isolation of the mRNA transcripts are well known in the art. Well known procedures include phenol/chloroform extraction or precipitation with either alcohol (ethanol, isopropanol) in the presence of monovalent cations or lithium chloride. Additional, non-limiting examples of purification procedures which can be used include size exclusion chromatography (Lukavsky, P. J. and Puglisi, J. D., 2004, Large-scale preparation and purification of polyacrylamide-free RNA oligonucleotides, RNA v.10, 889-893), silica-based affinity chromatography and polyacrylamide gel electrophoresis (Bowman, J. C., Azizi, B., Lenz, T. K., Ray, P., and Williams, L. D. in RNA in vitro transcription and RNA purification by denaturing PAGE in Recombinant and in vitro RNA syntheses Methods v. 941 Conn G. L. (ed), New York, N.Y. Humana Press, 2012). Purification can be performed using a variety of commercially available kits including, but not limited to SV Total Isolation System (Promega) and In Vitro Transcription Cleanup and Concentration Kit (Norgen Biotek).

Furthermore, while reverse transcription can yield large quantities of mRNA, the products can contain several aberrant RNA impurities associated with undesired polymerase activity which may need to be removed from the full-length mRNA preparation. These include short RNAs that result from abortive transcription initiation as well as double-stranded RNA (dsRNA) generated by RNA-dependent RNA polymerase activity, RNA-primed transcription from RNA templates and self-complementary 3′-extension. It has been demonstrated that these contaminants with dsRNA structures can lead to undesired immunostimulatory activity through interaction with various innate immune sensors in eukaryotic cells that function to recognize specific nucleic acid structures and induce potent immune responses. This in turn, can dramatically reduce mRNA translation since protein synthesis is reduced during the innate cellular immune response. Therefore, additional techniques to remove these dsRNA contaminants have been developed and are known in the art including but not limited to scaleable HPLC purification (see, e.g., Kariko, K., Muramatsu, H., Ludwig, J. And Weissman, D., 2011, Generating the optimal mRNA for therapy: HPLC purification eliminates immune activation and improves translation of nucleoside-modified, protein-encoding mRNA, Nucl Acid Res, v. 39 e142; Weissman, D., Pardi, N., Muramatsu, H., and Kariko, K., HPLC Purification of in vitro transcribed long RNA in Synthetic Messenger RNA and Cell Metabolism Modulation in Methods in Molecular Biology v.969 (Rabinovich, P. H. Ed), 2013). HPLC purified mRNA has been reported to be translated at much greater levels, particularly in primary cells and in vivo.

A significant variety of modifications have been described in the art which are used to alter specific properties of in vitro transcribed mRNA and improve its utility. These include but are not limited to modifications to the 5′- and 3′-termini of the mRNA. Endogenous eukaryotic mRNA typically contains a cap structure on the 5′-end of a mature molecule which plays an important role in mediating binding of the mRNA Cap Binding Protein (CBP), which is in turn responsible for enhancing mRNA stability in the cell and efficiency of mRNA translation. Therefore, highest levels of protein expression are achieved with capped mRNA transcripts. The 5′-cap contains a 5′-5′-triphosphate linkage between the 5′-most nucleotide and guanine nucleotide. The conjugated guanine nucleotide is methylated at the N7 position. Additional modifications include methylation of the ultimate and penultimate most 5′-nucleotides on the 2′-hydroxyl group.

Multiple distinct cap structures can be used to generate the 5′-cap of in vitro transcribed synthetic mRNA. 5′-capping of synthetic mRNA can be performed co-transcriptionally with chemical cap analogs (i.e., capping during in vitro transcription). For example, the Anti-Reverse Cap Analog (ARCA) cap contains a 5′-5′-triphosphate guanine-guanine linkage where one guanine contains an N7 methyl group as well as a 3′-O-methyl group. However, up to 20% of transcripts remain uncapped during this co-transcriptional process and the synthetic cap analog is not identical to the 5′-cap structure of an authentic cellular mRNA, potentially reducing translatability and cellular stability. Alternatively, synthetic mRNA molecules may also be enzymatically capped post-transcriptionally. These may generate a more authentic 5′-cap structure that more closely mimics, either structurally or functionally, the endogenous 5′-cap which have enhanced binding of cap binding proteins, increased half-life, reduced susceptibility to 5′ endonucleases and/or reduced 5′ de-capping. Numerous synthetic 5′-cap analogs have been developed and are known in the art to enhance mRNA stability and translatability (see, e.g., Grudzien-Nogalska, E., Kowalska, J., Su, W., Kuhn, A. N., Slepenkov, S. V., Darynkiewicz, E., Sahin, U., Jemielity, J., and Rhoads, R. E., Synthetic mRNAs with superior translation and stability properties in Synthetic Messenger RNA and Cell Metabolism Modulation in Methods in Molecular Biology v.969 (Rabinovich, P. H. Ed), 2013).

On the 3′-terminus, a long chain of adenine nucleotides (poly-A tail) is normally added to mRNA molecules during RNA processing. Immediately after transcription, the 3′ end of the transcript is cleaved to free a 3′ hydroxyl to which poly-A polymerase adds a chain of adenine nucleotides to the RNA in a process called polyadenylation. The poly-A tail has been extensively shown to enhance both translational efficiency and stability of mRNA (see Bernstein, P. and Ross, J., 1989, Poly(A), poly(A) binding protein and the regulation of mRNA stability, Trends Bio Sci v. 14 373-377; Guhaniyogi, J. And Brewer, G., 2001, Regulation of mRNA stability in mammalian cells, Gene, v. 265, 11-23; Dreyfus, M. And Regnier, P., 2002, The poly (A) tail of mRNAs: Bodyguard in eukaryotes, scavenger in bacteria, Cell, v.111, 611-613).

Poly (A) tailing of in vitro transcribed mRNA can be achieved using various approaches including, but not limited to, cloning of a poly (T) tract into the DNA template or by post-transcriptional addition using Poly (A) polymerase. The first case allows in vitro transcription of mRNA with poly (A) tails of defined length, depending on the size of the poly (T) tract, but requires additional manipulation of the template. The latter case involves the enzymatic addition of a poly (A) tail to in vitro transcribed mRNA using poly (A) polymerase which catalyzes the incorporation of adenine residues onto the 3′-termini of RNA, requiring no additional manipulation of the DNA template, but results in mRNA with poly(A) tails of heterogenous length. 5′-capping and 3′-poly (A) tailing can be performed using a variety of commercially available kits including, but not limited to Poly (A) Polymerase Tailing kit (EpiCenter), mMESSAGE mMACHINE T7 Ultra kit and Poly (A) Tailing kit (Life Technologies) as well as with commercially available reagents, various ARCA caps, Poly (A) polymerase, etc.

In addition to 5′-cap and 3′-poly adenylation, other modifications of the in vitro transcripts have been reported to provide benefits as related to efficiency of translation and stability. It is well known in the art that pathogenic DNA and RNA can be recognized by a variety of sensors within eukaryotes and trigger potent innate immune responses. The ability to discriminate between pathogenic and self-DNA and RNA has been shown to be based, at least in part, on structure and nucleoside modifications since most nucleic acids from natural sources contain modified nucleosides. In contrast, in vitro synthesized RNA lacks these modifications, thus rendering it immunostimulatory which in turn can inhibit effective mRNA translation as outlined above. The introduction of modified nucleosides into in vitro transcribed mRNA can be used to prevent recognition and activation of RNA sensors, thus mitigating this undesired immunostimulatory activity and enhancing translation capacity (see, e.g., Kariko, K. And Weissman, D. 2007, Naturally occurring nucleoside modifications suppress the immunostimulatory activity of RNA: implication for therapeutic RNA development, Curr Opin Drug Discov Devel, v.10 523-532; Pardi, N., Muramatsu, H., Weissman, D., Kariko, K., In vitro transcription of long RNA containing modified nucleosides in Synthetic Messenger RNA and Cell Metabolism Modulation in Methods in Molecular Biology v.969 (Rabinovich, P. H. Ed), 2013); Kariko, K., Muramatsu, H., Welsh, F. A., Ludwig, J., Kato, H., Akira, S., Weissman, D., 2008, Incorporation of Pseudouridine Into mRNA Yields Superior Nonimmunogenic Vector With Increased Translational Capacity and Biological Stability, Mol Ther v.16, 1833-1840. The modified nucleosides and nucleotides used in the synthesis of modified RNAs can be prepared monitored and utilized using general methods and procedures known in the art. A large variety of nucleoside modifications are available that may be incorporated alone or in combination with other modified nucleosides to some extent into the in vitro transcribed mRNA (see, e.g., US Publication No. 2012/0251618). In vitro synthesis of nucleoside-modified mRNA has been reported to have reduced ability to activate immune sensors with a concomitant enhanced translational capacity.

Other components of mRNA which can be modified to provide benefit in terms of translatability and stability include the 5′- and 3′-untranslated regions (UTR). Optimization of the UTRs (favorable 5′- and 3′-UTRs can be obtained from cellular or viral RNAs), either both or independently, have been shown to increase mRNA stability and translational efficiency of in vitro transcribed mRNA (see, e.g., Pardi, N., Muramatsu, H., Weissman, D., Kariko, K., In vitro transcription of long RNA containing modified nucleosides in Synthetic Messenger RNA and Cell Metabolism Modulation in Methods in Molecular Biology v.969 (Rabinovich, P. H. Ed), 2013).

In addition to mRNA, other nucleic acid payloads may be used for this disclosure. For oligonucleotides, methods of preparation include but are not limited to chemical synthesis and enzymatic, chemical cleavage of a longer precursor, in vitro transcription as described above, etc.

Methods of synthesizing DNA and RNA nucleotides are widely used and well known in the art (see, e.g., Gait, M. J. (ed.)Oligonucleotide synthesis: a practical approach, Oxford [Oxfordshire], Washington, D.C.: IRL Press, 1984; and Herdewijn, P. (ed.) Oligonucleotide synthesis: methods and applications, Methods in Molecular Biology, v. 288 (Clifton, N.J.) Totowa, N.J.: Humana Press, 2005; both of which are incorporated herein by reference).

For plasmid DNA, preparation for use with this disclosure commonly utilizes but is not limited to expansion and isolation of the plasmid DNA in vitro in a liquid culture of bacteria containing the plasmid of interest. The presence of a gene in the plasmid of interest that encodes resistance to a particular antibiotic (penicillin, kanamycin, etc.) allows those bacteria containing the plasmid of interest to selective grow in antibiotic-containing cultures. Methods of isolating plasmid DNA are widely used and well known in the art (see, e.g., Heilig, J., Elbing, K. L. and Brent, R (2001) Large-Scale Preparation of Plasmid DNA. Current Protocols in Molecular Biology. 41:11:1.7:1.7.1-1.7.16; Rozkov, A., Larsson, B., Gillström, S., Björnestedt, R. and Schmidt, S. R. (2008), Large-scale production of endotoxin-free plasmids for transient expression in mammalian cell culture. Biotechnol. Bioeng., 99: 557-566; and U.S. Pat. No. 6,197,553). Plasmid isolation can be performed using a variety of commercially available kits including, but not limited to Plasmid Plus (Qiagen), GenJET plasmid MaxiPrep (Thermo) and PureYield MaxiPrep (Promega) kits as well as with commercially available reagents.

Various exemplary embodiments of the cationic lipids of the present disclosure, lipid nanoparticles and compositions comprising the same, and their use to deliver active or therapeutic agents such as nucleic acids to modulate gene and protein expression, are described in further detail below.

As used herein, the following terms have the meanings ascribed to them unless specified otherwise.

Unless the context requires otherwise, throughout the present specification and claims, the word “comprise” and variations thereof, such as, “comprises” and “comprising” are to be construed in an open and inclusive sense, that is, as “including, but not limited to”.

Reference throughout this specification to “one embodiment” or “an embodiment” means that a particular feature, structure, or characteristic described in connection with the embodiment is included in at least one embodiment of the present disclosure. Thus, the appearances of the phrases “in one embodiment” or “in an embodiment” in various places throughout this specification are not necessarily all referring to the same embodiment. Furthermore, the features, structures, or characteristics may be combined in any suitable manner in one or more embodiments.

Unless defined otherwise, all technical and scientific terms used herein have the same meaning as is commonly understood by one of skill in the art to which this disclosure belongs. As used in the specification and claims, the singular form “a”, “an” and “the” include plural references unless the context clearly dictates otherwise.

The phrase “induce expression of a desired protein” refers to the ability of a nucleic acid to increase expression of the desired protein. To examine the extent of protein expression, a test sample (e.g., a sample of cells in culture expressing the desired protein) or a test mammal (e.g., a mammal such as a human or an animal model such as a rodent (e.g., mouse) or a non-human primate (e.g., monkey) model) is contacted with a nucleic acid (e.g., nucleic acid in combination with a lipid of the present disclosure). Expression of the desired protein in the test sample or test animal is compared to expression of the desired protein in a control sample (e.g., a sample of cells in culture expressing the desired protein) or a control mammal (e.g., a mammal such as a human or an animal model such as a rodent (e.g., mouse) or non-human primate (e.g., monkey) model) that is not contacted with or administered the nucleic acid. When the desired protein is present in a control sample or a control mammal, the expression of a desired protein in a control sample or a control mammal may be assigned a value of 1.0. In some embodiments, inducing expression of a desired protein is achieved when the ratio of desired protein expression in the test sample or the test mammal to the level of desired protein expression in the control sample or the control mammal is greater than 1, for example, about 1.1, 1.5, 2.0. 5.0 or 10.0. When a desired protein is not present in a control sample or a control mammal, inducing expression of a desired protein is achieved when any measurable level of the desired protein in the test sample or the test mammal is detected. One of ordinary skill in the art will understand appropriate assays to determine the level of protein expression in a sample, for example dot blots, northern blots, in situ hybridization, ELISA, immunoprecipitation, enzyme function, and phenotypic assays, or assays based on reporter proteins that can produce fluorescence or luminescence under appropriate conditions.

The term “nucleic acid” as used herein refers to a polymer containing at least two deoxyribonucleotides or ribonucleotides in either single- or double-stranded form and includes DNA, RNA, and hybrids thereof. DNA may be in the form of antisense molecules, plasmid DNA, cDNA, PCR products, or vectors. RNA may be in the form of small hairpin RNA (shRNA), messenger RNA (mRNA), antisense RNA, miRNA, micRNA, multivalent RNA, dicer substrate RNA or viral RNA (vRNA), and combinations thereof. Nucleic acids include nucleic acids containing known nucleotide analogs or modified backbone residues or linkages, which are synthetic, naturally occurring, and non-naturally occurring, and which have similar binding properties as the reference nucleic acid. Examples of such analogs include, without limitation, phosphorothioates, phosphoramidates, methyl phosphonates, chiral-methyl phosphonates, 2′-O-methyl ribonucleotides, and peptide-nucleic acids (PNAs). Unless specifically limited, the term encompasses nucleic acids containing known analogues of natural nucleotides that have similar binding properties as the reference nucleic acid. Unless otherwise indicated, a particular nucleic acid sequence also implicitly encompasses conservatively modified variants thereof (e.g., degenerate codon substitutions), alleles, orthologs, single nucleotide polymorphisms, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); Rossolini et al., Mol. Cell. Probes, 8:91-98 (1994)). “Nucleotides” contain a sugar deoxyribose (DNA) or ribose (RNA), a base, and a phosphate group. Nucleotides are linked together through the phosphate groups. “Bases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkylhalides.

The term “gene” refers to a nucleic acid (e.g., DNA or RNA) sequence that comprises partial length or entire length coding sequences necessary to produce a polypeptide or precursor polypeptide.

“Gene product,” as used herein, refers to a product of a gene such as an RNA transcript or a polypeptide.

The term “lipid” refers to a group of organic compounds that include, but are not limited to, esters of fatty acids and are generally characterized by being poorly soluble in water, but soluble in many organic solvents. They are usually divided into at least three classes: (1) “simple lipids,” which include fats and oils as well as waxes; (2) “compound lipids,” which include phospholipids and glycolipids; and (3) “derived lipids” such as steroids.

A “steroid” is a compound comprising the following carbon skeleton:

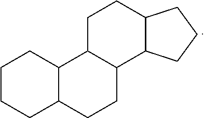 [SMILES: C1CCC2C(C1)CCC1C3CCCC3CCC21 ; "."]

Non-limiting examples of steroids include cholesterol, and the like.

A “cationic lipid” refers to a lipid capable of being positively charged. Exemplary cationic lipids include one or more amine group(s) which bear the positive charge. Preferred cationic lipids are ionizable such that they can exist in a positively charged or neutral form depending on pH. The ionization of the cationic lipid affects the surface charge of the lipid nanoparticle under different pH conditions. This charge state can influence plasma protein absorption, blood clearance and tissue distribution (Semple, S. C., et al., Adv. Drug Deliv Rev 32:3-17 (1998)) as well as the ability to form endosomolytic non-bilayer structures (Hafez, I. M., et al., Gene Ther 8:1188-1196 (2001)) critical to the intracellular delivery of nucleic acids.

The term “lipid nanoparticle” or “LNP” refers to particles having at least one dimension on the order of nanometers (e.g., 1-1,000 nm) which include one or more of the compounds of Formula (I) or other specified cationic lipids. In some embodiments, lipid nanoparticles are included in a formulation that can be used to deliver an active agent or therapeutic agent, such as a nucleic acid (e.g., mRNA) to a target site of interest (e.g., cell, tissue, organ, tumor, and the like). In some embodiments, the lipid nanoparticles of the disclosure comprise a nucleic acid. Such lipid nanoparticles typically comprise a compound of Formula (I) and one or more components selected from neutral lipids, charged lipids, steroids, and polymer conjugated lipids. In some embodiments, the active agent or therapeutic agent, such as a nucleic acid, may be encapsulated in the lipid portion of the lipid nanoparticle or an aqueous space enveloped by some or all the lipid portion of the lipid nanoparticle, thereby protecting it from enzymatic degradation or other undesirable effects induced by the mechanisms of the host organism or cells, e.g., an adverse immune response.

In various embodiments, the lipid nanoparticles have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm, and are substantially non-toxic. In certain embodiments, nucleic acids, when present in the lipid nanoparticles, are resistant in aqueous solution to degradation with a nuclease. Lipid nanoparticles comprising nucleic acids and their method of preparation are disclosed in, e.g., U.S. Patent Publication Nos. 2004/0142025, 2007/0042031 and PCT Pub. Nos. WO 2013/016058 and WO 2013/086373, the full disclosures of which are herein incorporated by reference in their entirety for all purposes.

As used herein, “lipid encapsulated” refers to a lipid nanoparticle that provides an active agent or therapeutic agent, such as a nucleic acid (e.g., mRNA), with full encapsulation, partial encapsulation, or both. In an embodiment, the nucleic acid (e.g., mRNA) is fully encapsulated in the lipid nanoparticle.

The term “polymer conjugated lipid” refers to a molecule comprising both a lipid portion and a polymer portion. An example of a polymer conjugated lipid is a pegylated lipid. The term “pegylated lipid” refers to a molecule comprising both a lipid portion and a polyethylene glycol portion. Pegylated lipids are known in the art and include 1-(monomethoxy-polyethyleneglycol)-2,3-dimyristoylglycerol (PEG-DMG) and the like.

The term “neutral lipid” refers to any of several lipid species that exist either in an uncharged or neutral zwitterionic form at a selected pH. At physiological pH, such lipids include, but are not limited to, phosphotidylcholines such as 1,2-Distearoyl-sn-glycero-3-phosphocholine (DSPC), 1,2-Dipalmitoyl-sn-glycero-3-phosphocholine (DPPC), 1,2-Dimyristoyl-sn-glycero-3-phosphocholine (DMPC), 1-Palmitoyl-2-oleoyl-sn-glycero-3-phosphocholine (POPC), 1,2-dioleoyl-sn-glycero-3-phosphocholine (DOPC), phophatidylethanolamines such as 1,2-Dioleoyl-sn-glycero-3-phosphoethanolamine (DOPE), sphingomyelins (SM), ceramides, steroids such as sterols and their derivatives. Neutral lipids may be synthetic or naturally derived.

The term “charged lipid” refers to any of a number of lipid species that exist in either a positively charged or negatively charged form independent of the pH within a useful physiological range, e.g., pH ˜3 to pH ˜9. Charged lipids may be synthetic or naturally derived. Examples of charged lipids include phosphatidylserines, phosphatidic acids, phosphatidylglycerols, phosphatidylinositols, sterol hemisuccinates, dialkyl trimethylammonium-propanes, (e.g., DOTAP, DOTMA), dialkyl dimethylaminopropanes, ethyl phosphocholines, dimethylaminoethane carbamoyl sterols (e.g., DC-Chol).

As used herein, the term “aqueous solution” refers to a composition comprising water.

“Alkyl” refers to a straight or branched hydrocarbon chain radical consisting solely of carbon and hydrogen atoms that is saturated (i.e., contains no double and/or triple bonds), having from one to twenty-four carbon atoms (C₁-C₂₄ alkyl), one to sixteen carbon atoms (C₁-C₁6 alkyl), one to twelve carbon atoms (C₁-C₁₂ alkyl), six to twenty-four carbon atoms (C₆-C₂₄ alkyl), one to eight carbon atoms (C₁-C₅ alkyl) or one to six carbon atoms (C₁-C₆ alkyl) and which is attached to the rest of the molecule by a single bond, e.g., methyl, ethyl, n-propyl, 1-methylethyl (iso propyl), n-butyl, n-pentyl, 1,1-dimethylethyl (t-butyl), 3-methylhexyl, 2-methylhexyl, and the like. Unless stated otherwise specifically in the specification, an alkyl group is optionally substituted.

“Alkenyl” refers to a straight or branched hydrocarbon chain radical consisting solely of carbon and hydrogen atoms that contains at least one carbon-carbon double, having from two to twenty-four carbon atoms (C₂-C₂₄ alkenyl), two to twelve carbon atoms (C₂-C₁₂ alkenyl), six to twenty-four carbon atoms (C₆-C₂₄ alkenyl), two to sixteen carbon atoms (C₂-C₁₆ alkenyl), four to twelve carbon atoms (C₄-C₁₂ alkenyl), two to eight carbon atoms (C₂-C₅ alkenyl), or two to six carbon atoms (C₂-C₆ alkenyl) and which is attached to the rest of the molecule by a single bond, e.g., ethenyl, n-propenyl, 1-methylethenyl, n-butenyl, n-pentenyl, 1,1-dimethylethenyl, 3-methylhexenyl, 2-methylhexenyl, and the like. Unless stated otherwise specifically in the specification, an alkenyl group is optionally substituted.

“Alkynyl” refers to a straight or branched hydrocarbon chain radical consisting solely of carbon and hydrogen atoms that contains at least one carbon-carbon triple bond, having from two to twenty-four carbon atoms (C₂-C₂₄ alkynyl), two to twelve carbon atoms (C₂-C₁₂ alkynyl), two to eight carbon atoms (C₂-C₅ alkynyl), or two to six carbon atoms (C₂-C₆ alkynyl) and which is attached to the rest of the molecule by a single bond, e.g., ethynyl, n-propynyl, 1-methylethynyl, n-butynyl, n-pentynyl, 1,1-dimethylethynyl, 3-methylhexynyl, 2-methylhexynyl, and the like. Unless stated otherwise specifically in the specification, an alkynyl group is optionally substituted.

“Alkylene” or “alkylene chain” refers to a straight or branched divalent saturated hydrocarbon chain linking the rest of the molecule to a radical group, consisting solely of carbon and hydrogen. In some embodiments, an alkylene chain has from one to twenty-four carbon atoms (C₁-C₂₄ alkylene), one to fifteen carbon atoms (C₁-C₁₅ alkylene), one to twelve carbon atoms (C₁-C₁₂ alkylene), one to eight carbon atoms (C₁-C₅ alkylene), one to six carbon atoms (C₁-C₆ alkylene), four to six carbon atoms (C₄-C₆ alkylene), two to four carbon atoms (C₂-C₄ alkylene), one to two carbon atoms (C₁-C₂ alkylene), e.g., methylene, ethylene, propylene, n-butylene, and the like. The alkylene chain is attached to the rest of the molecule through a single bond and to the radical group through a single bond. The points of attachment of the alkylene chain to the rest of the molecule and to the radical group can be through one carbon or any two carbons within the chain. Unless stated otherwise specifically in the specification, an alkylene chain is optionally substituted.

“Alkoxy” refers to a radical having the following formula: —O—R^{a} wherein R^{a} is an alkyl radical as defined above. Unless stated otherwise specifically in the specification, the alkyl portion of the radical is optionally substituted.

“Halo” refers to a halogen substituent (i.e., F, Cl, Br, or I).

“Cyano” refers to a —CN functional group.

The term “substituted” used herein means any of the above groups (e.g., alkyl, alkylhydroxyl, alkenyl, alkynyl, alkylene, cycloalkyl, aryl, aralkyl or heterocyclyl) wherein at least one hydrogen atom is replaced by a bond to a non-hydrogen atoms such as, but not limited to: a halogen atom such as F, Cl, Br, and I; oxo groups (═O); hydroxyl groups (—OH); alkoxy groups (—OR^{a}, where R^{a} is C₁-C₁₂ alkyl or cycloalkyl); carboxyl groups (—OC(═O)R^{a} or —C(═O)OR^{a}, where R^{a} is H, C₁-C₁₂ alkyl or cycloalkyl); amine groups (—NR^{a}R^{b}, where R^{a} and R^{b} are each independently H, C₁-C₁₂ alkyl or cycloalkyl); C₁-C₁₂ alkyl groups; and cycloalkyl groups. In some embodiments the substituent is a C₁-C₁₂ alkyl group. In other embodiments, the substituent is a cycloalkyl group. In other embodiments, the substituent is a halo group, such as fluoro. In other embodiments, the substituent is an oxo group. In other embodiments, the substituent is a hydroxyl group. In other embodiments, the substituent is an alkoxy group. In other embodiments, the substituent is a carboxyl group. In other embodiments, the substituent is an amine group.

“Optional” or “optionally” (e.g., optionally substituted) means that the subsequently described event of circumstances may or may not occur, and that the description includes instances where said event or circumstance occurs and instances in which it does not. For example, “optionally substituted alkyl” means that the alkyl radical may or may not be substituted and that the description includes both substituted alkyl radicals and alkyl radicals having no substitution. In some embodiments, “optionally substituted” means a particular radical is substituted with one or more substituents selected from the group consisting of halo (e.g., F, Cl, Br, and I), oxo (═O), hydroxyl (—OH), alkoxy (—OR^{a}, where R^{a} is C₁-C₁₂ alkyl), cycloalkoxy (—OR^{a}, where R^{a} is C₃-C₈ cycloalkyl), carboxyl (—OC(═O)R^{a} or —C(═O)OR^{a}, where R^{a} is H, C₁-C₁₂ alkyl, or C₃-C₈ cycloalkyl), amine (—NR^{a}R^{b}, where R^{a} and R^{b} are each independently H, C₁-C₁₂ alkyl, or C₃-C₈ cycloalkyl), C₁-C₁₂ alkyl, and C₃-C₈ cycloalkyl.

In some embodiments, “optionally substituted” means substituted with one or more halo substituents. In some embodiments, “optionally substituted” means substituted with one or more oxo substituents. In some embodiments, “optionally substituted” means substituted with one or more hydroxyl substituents. In certain embodiments, “optionally substituted” means substituted with one or more alkoxy substituents. In some embodiments, “optionally substituted” means substituted with one or more cycloalkoxy substituents. In certain embodiments, “optionally substituted” means substituted with one or more carboxy substituents. In some embodiments, “optionally substituted” means substituted with one or more amine substituents. In certain embodiments, “optionally substituted” means substituted with one or more C₁-C₁₂ alkyl substituents. In some embodiments, “optionally substituted” means substituted with one or more C₃-C₅ cycloalkyl substituents.

When a functional group is described as “optionally substituted,” and in turn, substituents on the functional group are also “optionally substituted” and so on, for the purposes of this disclosure, such iterations are limited to five, preferably such iterations are limited to two. In some embodiments, such iterations are limited to one. In some embodiments, such iterations are limited to zero.

This disclosure is also meant to encompass all pharmaceutically acceptable compounds of Formula (I) being isotopically labelled by having one or more atoms replaced by an atom having a different atomic mass or mass number. Examples of isotopes that can be incorporated into the disclosed compounds include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. These radiolabeled compounds could be useful to help determine or measure the effectiveness of the compounds, by characterizing, for example, the site or mode of action, or binding affinity to pharmacologically important site of action. Certain isotopically labelled compounds of Formula (I), for example, those incorporating a radioactive isotope, are useful in drug and/or substrate tissue distribution studies. The radioactive isotopes tritium, i.e., ³H, and carbon-14, i.e., ¹⁴C, are particularly useful for this purpose in view of their ease of incorporation and ready means of detection.

Substitution with heavier isotopes such as deuterium, i.e., ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy.

Isotopically labeled compounds of Formula (I) can generally be prepared by conventional techniques known to those skilled in the art or by processes analogous to those described in the Preparations and Examples as set out below using an appropriate isotopically labeled reagent in place of the non-labeled reagent previously employed.

This disclosure is also meant to encompass the in vivo metabolic products of the disclosed compounds. Such products may result from, for example, the oxidation, reduction, hydrolysis, amidation, esterification, and the like of the administered compound, primarily due to enzymatic processes. Accordingly, the disclosure includes compounds produced by a process comprising administering a compound of this disclosure to a mammal for a period sufficient to yield a metabolic product thereof. Such products are typically identified by administering a radiolabeled compound of the disclosure in a detectable dose to an animal, such as rat, mouse, guinea pig, monkey, or to human, allowing sufficient time for metabolism to occur, and isolating its conversion products from the urine, blood, or other biological samples.

“Stable compound” and “stable structure” are meant to indicate a compound that is sufficiently robust to survive isolation to a useful degree of purity from a reaction mixture, and formulation into an efficacious therapeutic agent.

“Mammal” includes humans and both domestic animals such as laboratory animals and household pets (e.g., cats, dogs, swine, cattle, sheep, goats, horses, rabbits), and non-domestic animals such as wildlife and the like.

“Pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Pharmaceutically acceptable salt” includes both acid and base addition salts.

“Pharmaceutically acceptable acid addition salt” refers to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

Often crystallizations produce a solvate of the compound of the disclosure. As used herein, the term “solvate” refers to an aggregate that comprises one or more molecules of a compound of the disclosure with one or more molecules of solvent. The solvent may be water, in which case the solvate may be a hydrate. Alternatively, the solvent may be an organic solvent. Thus, the compounds of the present disclosure may exist as a hydrate, including a monohydrate, dihydrate, hemihydrate, sesquihydrate, trihydrate, tetrahydrate and the like, as well as the corresponding solvated forms. The compound of the disclosure may be true solvates, while in other cases, the compound of the disclosure may merely retain adventitious water or be a mixture of water plus some adventitious solvent.

A “pharmaceutical composition” refers to a formulation of a compound of the disclosure and a medium generally accepted in the art for the delivery of the biologically active compound to mammals, e.g., humans. Such a medium includes all pharmaceutically acceptable carriers, diluents, or excipients therefor.

An “effective amount” or “therapeutically effective amount” of an active agent or therapeutic agent such as a therapeutic nucleic acid is an amount sufficient to produce the desired effect, e.g., an increase or inhibition of expression of a target sequence in comparison to the normal expression level detected in the absence of the nucleic acid. An increase in expression of a target sequence is achieved when any measurable level is detected in the case of an expression product that is not present in the absence of the nucleic acid. In the case where the expression product is present at some level prior to contact with the nucleic acid, an in increase in expression is achieved when the fold increase in value obtained with a nucleic acid such as mRNA relative to control is about 1.05, 1.1, 1.2, 1.3, 1.4, 1.5, 1.75, 2, 2.5, 3, 4, 5, 6, 7, 8, 9, 10, 15, 20, 25, 30, 40, 50, 75, 100, 250, 500, 750, 1000, 5000, 10000 or greater. Inhibition of expression of a target gene or target sequence is achieved when the value obtained with a nucleic acid such as antisense oligonucleotide relative to the control is about 95%, 90%, 85%, 80%, 75%, 70%, 65%, 60%, 55%, 50%, 45%, 40%, 35%, 30%, 25%, 20%, 15%, 10%, 5%, or 0%. Suitable assays for measuring expression of a target gene or target sequence include, e.g., examination of protein or RNA levels using techniques known to those of skill in the art such as dot blots, northern blots, in situ hybridization, ELISA, immunoprecipitation, enzyme function, fluorescence or luminescence of suitable reporter proteins, as well as phenotypic assays known to those of skill in the art.

“Treating” or “treatment” as used herein covers the treatment of the disease or condition of interest in a mammal, preferably a human, having the disease or condition of interest, and includes:
(i) preventing the disease or condition from occurring in a mammal, in particular, when such mammal is predisposed to the condition but has not yet been diagnosed as having it;
(ii) inhibiting the disease or condition, i.e., arresting its development;
(iii) relieving the disease or condition, i.e., causing regression of the disease or condition; or
(iv) relieving the symptoms resulting from the disease or condition, i.e., relieving pain without addressing the underlying disease or condition. As used herein, the terms “disease” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians.

The compounds of the disclosure, or their pharmaceutically acceptable salts may contain one or more asymmetric centers and may thus give rise to enantiomers, diastereomers, and other stereoisomeric forms that may be defined, in terms of absolute stereochemistry, as (R)- or (S)—or, as (D)- or (L)- for amino acids. The present disclosure is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high-pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. The present disclosure contemplates various stereoisomers and mixtures thereof and includes “enantiomers”, which refers to two stereoisomers whose molecules are non-superimposable mirror images of one another.

A “tautomer” refers to a proton shift from one atom of a molecule to another atom of the same molecule. The present disclosure includes tautomers of any said compounds.

### Compounds

In an aspect, the disclosure provides novel lipid compounds which can combine with other components such as cationic lipids, neutral lipids, charged lipids, steroids, and/or polymer conjugated lipids to form lipid nanoparticles with oligonucleotides. Without wishing to be bound by theory, it is thought that these lipid nanoparticles shield oligonucleotides from degradation in the serum and provide for effective delivery of oligonucleotides to cells in vitro and in vivo.

One embodiment provides a compound having the following Formula (I):

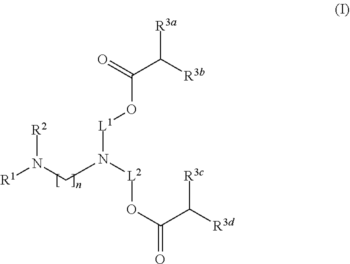

or a pharmaceutically acceptable salt, tautomer, or stereoisomer thereof, wherein:
R¹ and R² are each independently optionally substituted C₁-C₆ alkyl;
R^{3a}, R^{3b}, R^{3c}, and R^{3d} are each independently optionally substituted C₄-C₁₂ alkyl;
L¹ and L² are each independently C₃-C₅ alkylene;
n is 2, 3, 4, or 5.

In some embodiments, R¹ is unsubstituted. In certain other embodiments, R¹ is substituted with —OH, —NH₂, alkoxy, or halo.

In certain embodiments, R² is unsubstituted. In some embodiments, R² is substituted with —OH, —NH₂, alkoxy, or halo.

In some embodiments, R¹ is methyl, ethyl, isopropyl, or n-propyl. In certain embodiments, R¹ is methyl.

In certain embodiments, R² is methyl, ethyl, isopropyl, or n-propyl. In some embodiments, R² is methyl.

In some embodiments, R¹ and R² are both methyl.

In certain embodiments, L¹ is unsubstituted. In some embodiments, L² is unsubstituted. In some embodiments, L¹ is C₄ alkylene. In certain embodiments, L² is C₄ alkylene. In some embodiments, L¹ and L² are both C₄ alkylene.

In some embodiments, R^{3a} is C₆-C₁₀ alkyl. In certain embodiments, R^{3a} is C₈ alkyl. In some embodiments, R^{3b} is C₄-C₈ alkyl. In certain embodiments, R^{3b} is C₆ alkyl.

In certain embodiments, R^{3′} is C₆-C₁₀ alkyl. In some embodiments, R^{3c} is C₈ alkyl. In certain embodiments, R^{3d} is C₄-C₈ alkyl. In some embodiments, R^{3d} is C₆ alkyl.

In some embodiments, R^{3a} is C₁₀-C₁₂ alkyl. In certain embodiments, R^{3a} is C₁₀ alkyl. In some embodiments, R^{3b} is C₁₀-C₁₂ alkyl. In certain embodiments, R^{3b} is C₁₂ alkyl. In some embodiments, R^{3c} is C₁₀-C₁₂ alkyl. In certain embodiments, R³, is C₁₀ alkyl. In some embodiments, R^{3d} is C₁₀-C₁₂ alkyl.

In some embodiments, at least one of R^{3a}, R^{3b}, R^{3c}, or R^{3d} is unsubstituted. In certain embodiments, at least one of R^{3a}, R^{3b}, R^{3c}, or R^{3d} is substituted with one or more substituents selected from the group consisting of —OH, —NH₂, alkoxy, cyano, and halo. In some embodiments, R^{3a}, R^{3b}, R^{3c}, and R^{3d} are unsubstituted.

In certain embodiments, R^{3a} and R^{3c} are C₈ alkyl and R^{3b} and R^{3d} are C₆ alkyl. In certain embodiments, R^{3d} is C₁₂ alkyl. In some embodiments, R^{3a} and R^{3c} are C₁₀ alkyl and R^{3b} and R^{3d} are C₁₂ alkyl.

In various embodiments, the compound or a pharmaceutically acceptable salt, tautomer, hydrate, solvate, stereoisomer, or isotope thereof has one of the structures set forth in Table 1 below.

TABLE 1
Representative compounds of Formula (I)

| No. | Compound Structure |
|---|---|
| I-1 | 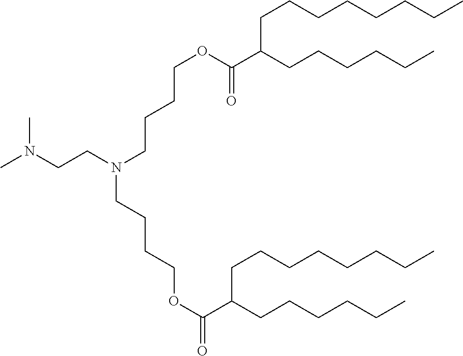 [SMILES: CCCCCCCCC(CCCCCC)C(=O)OCCCCN(CCCCOC(=O)C(CCCCCC)CCCCCCCC)CCN(C)C] |
| I-2 | 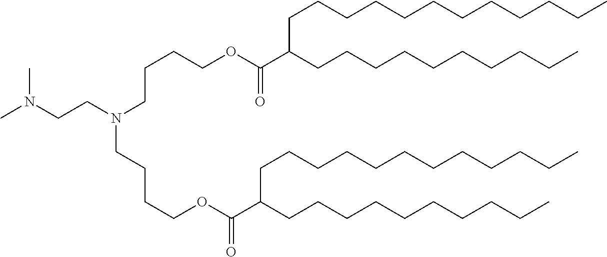 [SMILES: CCCCCCCCCCCCC(CCCCCCCCCC)C(=O)OCCCCN(CCCCOC(=O)C(CCCCCCCCCC)CCCCCCCCCCCC)CCN(C)C] |

It is understood that any embodiment of the compounds of Formula (I), as set forth above, and any specific substituent and/or variable in the compound Formula (I), as set forth above, may be independently combined with other embodiments and/or substituents and/or variables of compounds of Formula (I) to form embodiments of the disclosure not specifically set forth above. In addition, in the event that a list of substituents and/or variables is listed for any particular R group or L group in a particular embodiment and/or claim, it is understood that each individual substituent and/or variable may be deleted from the particular embodiment and/or claim and that the remaining list of substituents and/or variables will be considered to be within the scope of embodiments of the disclosure.

It is understood that in the present description, combinations of substituents and/or variables of the depicted formulae are permissible only if such contributions result in stable compounds.

For the purposes of administration, the compounds of the present disclosure (typically in the form of lipid nanoparticles in combination with a therapeutic agent) may be administered as a raw chemical or may be formulated as pharmaceutical compositions. Pharmaceutical compositions of the present disclosure comprise a compound of Formula (I) and one or more pharmaceutically acceptable carrier, diluent, or excipient. The compound of Formula (I) is present in the composition in an amount which is effective to form a lipid nanoparticle and deliver the therapeutic agent, e.g., for treating a particular disease or condition of interest. Appropriate concentrations and dosages can be readily determined by one skilled in the art.

One embodiment provides a lipid nanoparticle that comprises a compound of Formula (I).

An embodiment provides a composition comprising a compound of Formula (I) and a therapeutic agent. In some embodiments, the composition further comprises one or more component selected from cationic lipids, neutral lipids, steroids, and polymer conjugated lipids.

In some embodiments, the therapeutic agent comprises a nucleic acid. In certain embodiments, the nucleic acid is selected from antisense and messenger RNA.

In some embodiments, the composition or lipid nanoparticle comprising compounds of Formula (I) further comprises additional cationic lipids. Exemplary cationic lipids and their synthesis can be found in the following publications:
U.S. Pat. Nos. 9,738,593; 10,221,127; 10,166,298; 11,357,856; 11,712,481; 11,453,639;
US Patent Publication Nos: US 2018/0185516; US 2022/0106257;
PCT Publication Nos. WO 2017/117528; WO 2016/176330; WO 2018/191719; WO 2018/200943; WO 2019/036000; WO 2019/036028; WO 2019/036030; WO 2019/036008; WO 2019/089828; WO 2020/061426; WO 2020/081938; WO 2021/030701; WO 2023/114944; WO 2023/114939; WO 2023/114943, the disclosures of which are hereby incorporated by reference.

In certain embodiments, the composition comprises one or more neutral lipids selected from DSPC, DPPC, DMPC, DOPC, POPC, DOPE and SM. In some embodiments, the neutral lipid is DSPC. In some embodiments, the molar ratio of the compound to the neutral lipid ranges from about 2:1 to about 8:1. In certain embodiments, the steroid is cholesterol. In some embodiments, the molar ratio of the compound to cholesterol ranges from about 2:1 to about 1:1. In certain embodiments, the molar ratio of the compound to cholesterol ranges from about 5:1 to about 1:1 or from about 2:1 to about 1:1.

In certain embodiments, the polymer conjugated lipid is a pegylated lipid. In various embodiments, the polymer conjugated lipid is a pegylated lipid. For example, some embodiments include a pegylated diacylglycerol (PEG-DAG) such as 1-(monomethoxy-polyethyleneglycol)-2,3-dimyristoylglycerol (PEG-DMG), a pegylated phosphatidylethanoloamine (PEG-PE), a PEG succinate diacylglycerol (PEG-S-DAG) such as 4-O-(2′,3′-di(tetradecanoyloxy)propyl-1-O-(ω-methoxy(polyethoxy)ethyl)butanedioate (PEG-S-DMG), a pegylated ceramide (PEG-cer), or a PEG dialkoxypropylcarbamate such as ω-methoxy(polyethoxy)ethyl-N-(2,3-di(tetradecanoxy)propyl)carbamate or 2,3-di(tetradecanoxy)propyl-N-(ω-methoxy(polyethoxy)ethyl)carbamate.

In some embodiments, the molar ratio of the compound to the pegylated lipid ranges from about 100:1 to about 10:1 or from about 100:1 to about 25:1. In some embodiments, the molar ratio of the compound to pegylated lipid ranges from about 100:1 to about 20:1 or from about 100:1 to about 10:1. In some embodiments, the pegylated lipid is PEG-DMG. In some embodiments, the pegylated lipid is PEG-DAG, PEG-PE, PEG-S-DAG, PEG-cer or a PEG dialkyoxypropylcarbamate.

In some embodiments, the lipid nanoparticle further comprises at least one pegylated lipid having a structure of Formula (II):

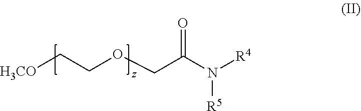

or a pharmaceutically acceptable salt, tautomer, hydrate, solvate, stereoisomer, or isotope thereof, wherein:
R⁴ and R⁵ are each independently a straight or branched alkyl, alkenyl, or alkynyl containing from 10 to 30 carbon atoms, wherein each alkyl, alkenyl, or alkynyl is optionally substituted with at least one fluoro; and
z is an integer ranging from 30 to 60.

In some embodiments, R⁴ and R⁵ are each independently straight, alkyl chains containing from 12 to 16 carbon atoms, wherein each alkyl is optionally substituted with at least one fluoro. In certain embodiments, R⁴ and R⁵ are each independently straight alkyl chains containing from 12 to 16 carbon atoms.

In some embodiments, R⁴ and R⁵ are each independently:

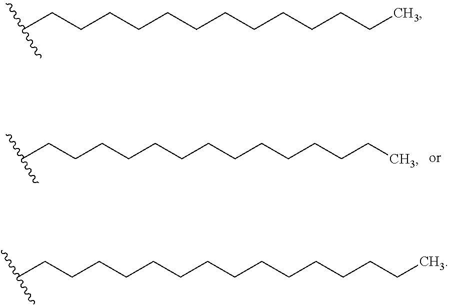 [SMILES: *CCCCCCCCCCCCC ; *CCCCCCCCCCCCCC ; "or" ; *CCCCCCCCCCCCCCC]

In some embodiments, wherein z is an integer ranging from 45 to 50. In some embodiments, wherein z is an integer ranging from 42 to 48. In some embodiments, the at least one pegylated lipid has the following structure:

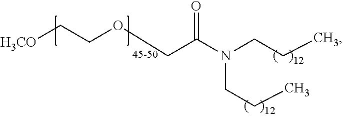

or a pharmaceutically acceptable salt, tautomer, hydrate, solvate, stereoisomer, or isotope thereof.

In some embodiments, the lipid nanoparticle or composition comprises a plurality of pegylated lipids of Formula (II). In some embodiments, the plurality of pegylated lipids has an average value of z ranging from 40 to 55. In some embodiments, the plurality of pegylated lipids has an average value of z ranging from 40 to 50, or 42 to 48. In some embodiments, the plurality of pegylated lipids has an average value of z ranging from 30 to 55, 30 to 50, 30 to 45, 30 to 40, or 30 to 35. In some embodiments, the plurality of lipids has an average value of z ranging from 35 to 55, 40 to 55, 42 to 55, 45 to 55, or 48 to 55.

Synthesis of pegylated lipids (e.g., compounds of Formula (II)) can be found in U.S. Pat. No. 9,738,593, the disclosure of which is hereby incorporated by reference.

It is understood that any embodiment of the compounds of Formula (I) as set forth above, and any specific substituent and/or variable in the compound Formula (I) as set forth above, may be independently combined with other embodiments and/or substituents and/or variables of compounds of Formula (I) to form embodiments of the disclosure not specifically set forth herein. In addition, in the event that a list of substituents and/or variables is listed for any particular R group or L group in a particular embodiment and/or claim, it is understood that each individual substituent and/or variable may be deleted from the particular embodiment and/or claim and that the remaining list of substituents and/or variables will be considered to be within the scope of embodiments of the disclosure.

It is understood that in the present description, combinations of substituents and/or variables of the depicted formulae are permissible only if such contributions result in stable compounds.

As used herein, “mol percent,” “mole percent,” or “mol %” refers to a component's molar percentage relative to the total number of mols of all components of a lipid nanoparticle excluding a therapeutic agent (i.e., total mols of cationic lipid(s), neutral lipid(s), steroid(s), and polymer conjugated lipid(s)).

In some embodiments, the cationic lipid (e.g., the compound of Formula (I)) is present at a concentration ranging from about 35 to about 70 mol % of the lipid nanoparticle. In some embodiments, the cationic lipid (e.g., the compound of Formula (I)) is present at a concentration ranging from about 35 to about 70 mol %, from about 40 to about 60 mol %, from about 45 to about 50 mol %, from about 45 to about 49 mol %, from about 40 to about 55 mol %, or from about 46 to about 48 mol % of the lipid nanoparticle.

In some embodiments, the concentration of the pegylated lipid (e.g., compounds of Formula (II)) ranges from about 1.0 to about 10.0 mol % of the lipid nanoparticle. In some embodiments, the concentration of the pegylated lipid (e.g., compounds of Formula (II)) ranges from about 1.3 to about 2.0 mol % of the lipid nanoparticle. In some embodiments, the concentration of the pegylated lipid (e.g., compounds of Formula (II)) is about 1.5. In certain embodiments, the concentration of the pegylated lipid (e.g., compounds of Formula (II)) is about 1.7.

In some embodiments, the lipid nanoparticle further comprises a cationic lipid. In some embodiments, the lipid nanoparticle further comprises a neutral lipid. In some embodiments, the lipid nanoparticle further comprises a steroid. In some embodiments, the lipid nanoparticle comprising a compound of Formula (I) further comprises a cationic lipid, a neutral lipid, a steroid, or a combination thereof.

One embodiment provides a lipid nanoparticle comprising at least one neutral lipid. In some embodiments, the at least one neutral lipid is selected from the group consisting of 1,2-Distearoyl-sn-glycero-3-phosphocholine (DSPC), 1,2-Dipalmitoyl-sn-glycero-3-phosphocholine (DPPC), 1,2-Dimyristoyl-sn-glycero-3-phosphocholine (DMPC), 1-Palmitoyl-2-oleoyl-sn-glycero-3-phosphocholine (POPC), 1,2-dioleoyl-sn-glycero-3-phosphocholine (DOPC), 1,2-Dioleoyl-sn-glycero-3-phosphoethanolamine (DOPE), a sphingomyelin (SM), and combinations thereof. In some embodiments, the at least one neutral lipid comprises 1,2-Distearoyl-sn-glycero-3-phosphocholine (DSPC).

In some embodiments, the neutral lipid is present at a concentration ranging from about 5 to about 15 mol % of the lipid nanoparticle. In some embodiments, the steroid is present at a concentration ranging from about 7 to about 13 mol %, or from about 9 to about 11 mol %.

In certain embodiments, the lipid nanoparticle comprises at least one steroid. In certain embodiments, the at least one steroid comprises cholesterol.

In some embodiments, the steroid is present at a concentration ranging from about 39 to about 49 mol % of the lipid nanoparticle. In some embodiments, the steroid is present at a concentration ranging from about 40 to about 50 mol %, from about 41 to about 49 mol %, or from about 46 to about 44 mol %.

In some embodiments, the lipid nanoparticle further comprises at least one therapeutic agent. In certain embodiments, the therapeutic agent comprises a nucleic acid. In some embodiments, the therapeutic agent is a nucleic acid. In certain embodiments, the nucleic acid comprises an antisense RNA, a messenger RNA, or a combination thereof. In some embodiments, the at least one therapeutic agent comprises Cas9 mRNA or ribonucleoprotein.

In certain embodiments, the lipid nanoparticle has a size of about 40 nm to about 70 nm. In some embodiments, the lipid nanoparticle has a size of about 45 nm to about 65 nm, about 50 nm to about 60 nm, about 30 nm to about 70 nm, about 35 nm to about 75 nm, about 45 nm to about 80 nm, about 25 nm to about 100 nm, about 20 nm to about 90 nm, about 15 nm to about 150 nm, or about 10 nm to about 200 nm.

One embodiment provides a pharmaceutical composition, comprising a lipid nanoparticle of the present disclosure and a pharmaceutically acceptable diluent or excipient.

Administration of the compositions of the disclosure can be carried out via any of the accepted modes of administration of agents for serving similar utilities. The pharmaceutical compositions of the disclosure may be formulated into preparations in solid, semi-solid, liquid, or gaseous forms, such as tablets, capsules, powders, granules, ointments, solutions, suspensions, suppositories, injections, inhalants, gels, microspheres, and aerosols. Typical routes of administering such pharmaceutical compositions include, without limitation, oral, topical, transdermal, inhalation, parenteral, sublingual, buccal, rectal, vaginal, and intranasal. The term parenteral as used herein includes subcutaneous injections, intravenous, intramuscular, intradermal, intrasternal injection or infusion techniques. Pharmaceutical compositions of the disclosure are formulated to allow the active ingredients contained therein to be bioavailable upon administration of the composition to a patient. Compositions that will be administered to a subject or patient take the form of one or more dosage units, where for example, a tablet may be a single dosage unit, and a container of a compound of the disclosure in aerosol form may hold a plurality of dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition to be administered will, in any event, contain a therapeutically effective amount of a compound of the disclosure, or a pharmaceutically acceptable salt thereof, for treatment of a disease or condition of interest in accordance with the teachings of this disclosure.

A pharmaceutical composition of the disclosure may be in the form of a solid or liquid. In one aspect, the carrier(s) are particulate, so that the compositions are, for example, in tablet or powder form. The carrier(s) may be liquid, with the compositions being, for example, an oral syrup, injectable liquid, or an aerosol, which is useful in, for example, inhalatory administration.

When intended for oral administration, the pharmaceutical composition is preferably in either solid or liquid form, where semi-solid, semi-liquid, suspension and gel forms are included within the forms considered herein as either solid or liquid.

As a solid composition for oral administration, the pharmaceutical composition may be formulated into a powder, granule, compressed tablet, pill, capsule, chewing gum, wafer, or the like form. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent.

When the pharmaceutical composition is in the form of a capsule, for example, a gelatin capsule, it may contain, in addition to materials of the above type, a liquid carrier such as polyethylene glycol or oil.

The pharmaceutical composition may be in the form of a liquid, for example, an elixir, syrup, solution, emulsion, or suspension. The liquid may be for oral administration or for delivery by injection, as two examples. When intended for oral administration, preferred composition contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer, and isotonic agent may be included.

The liquid pharmaceutical compositions of the disclosure, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose; agents to act as cryoprotectants such as sucrose or trehalose. The parenteral preparation can be enclosed in ampoules, disposable syringes or multiple dose vials made of glass or plastic.

Physiological saline is a preferred adjuvant. An injectable pharmaceutical composition is preferably sterile.

A liquid pharmaceutical composition of the disclosure intended for either parenteral or oral administration should contain an amount of a compound of the disclosure such that a suitable dosage will be obtained.

The pharmaceutical composition of the disclosure may be intended for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a pharmaceutical composition for topical administration. If intended for transdermal administration, the composition may include a transdermal patch or iontophoresis device.

The pharmaceutical composition of the disclosure may be intended for rectal administration, in the form, for example, of a suppository, which will melt in the rectum and release the drug. The composition for rectal administration may contain an oleaginous base as a suitable nonirritating excipient. Such bases include, without limitation, lanolin, cocoa butter, and polyethylene glycol.

The pharmaceutical composition of the disclosure may include various materials, which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule.

The pharmaceutical composition of the disclosure in solid or liquid form may include an agent that binds to the compound of the disclosure and thereby assists in the delivery of the compound. Suitable agents that may act in this capacity include a monoclonal or polyclonal antibody, or a protein.

The pharmaceutical composition of the disclosure may consist of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients.

Aerosols of compounds of the disclosure may be delivered in single phase, bi-phasic, or tri-phasic systems to deliver the active ingredient(s). Delivery of the aerosol includes the necessary container, activators, valves, sub-containers, and the like, which together may form a kit. One skilled in the art, without undue experimentation may determine preferred aerosols.

The pharmaceutical compositions of the disclosure may be prepared by methodology well known in the pharmaceutical art. For example, a pharmaceutical composition intended to be administered by injection can be prepared by combining the lipid nanoparticles of the disclosure with sterile, distilled water or other carrier to form a solution. A surfactant may be added to facilitate the formation of a homogeneous solution or suspension. Surfactants are compounds that non-covalently interact with the compound of the disclosure to facilitate dissolution or homogeneous suspension of the compound in the aqueous delivery system.

The compositions of the disclosure, or their pharmaceutically acceptable salts, are administered in a therapeutically effective amount, which will vary depending upon a variety of factors including the activity of the specific therapeutic agent employed; the metabolic stability and length of action of the therapeutic agent; the age, body weight, general health, sex, and diet of the patient; the mode and time of administration; the rate of excretion; the drug combination; the severity of the particular disorder or condition; and the subject undergoing therapy.

Compositions of the disclosure may also be administered simultaneously with, prior to, or after administration of one or more other therapeutic agents. Such combination therapy includes administration of a single pharmaceutical dosage formulation of a composition of the disclosure and one or more additional active agents, as well as administration of the composition of the disclosure and each active agent in its own separate pharmaceutical dosage formulation. For example, a composition of the disclosure and the other active agent can be administered to the patient together in a single oral dosage composition such as a tablet or capsule, or each agent administered in separate oral dosage formulations. Where separate dosage formulations are used, the compounds of the disclosure and one or more additional active agents can be administered at essentially the same time, i.e., concurrently, or at separately staggered times, i.e., sequentially; combination therapy is understood to include all these regimens.

Preparation methods for the above compounds and compositions are described herein below and/or known in the art.

It will be appreciated by those skilled in the art that in the process described herein the functional groups of intermediate compounds may need to be protected by suitable protecting groups. Such functional groups include hydroxy, amino, mercapto and carboxylic acid. Suitable protecting groups for hydroxy include trialkylsilyl or diarylalkylsilyl (for example, t-butyldimethylsilyl, t-butyldiphenylsilyl or trimethylsilyl), tetrahydropyranyl, benzyl, and the like.

Suitable protecting groups for amino, amidino and guanidino include t-butoxycarbonyl, benzyloxycarbonyl, and the like. Suitable protecting groups for mercapto include —C(O)—R″ (where R″ is alkyl, aryl or arylalkyl), p-methoxybenzyl, trityl and the like. Suitable protecting groups for carboxylic acid include alkyl, aryl or arylalkyl esters. Protecting groups may be added or removed in accordance with standard techniques, which are known to one skilled in the art and as described herein. The use of protecting groups is described in detail in Green, T. W. and P. G. M. Wutz, Protective Groups in Organic Synthesis (1999), 3^{rd} Ed., Wiley. As one of skill in the art would appreciate, the protecting group may also be a polymer resin such as a Wang resin, Rink resin or a 2-chlorotrityl-chloride resin.

It will also be appreciated by those skilled in the art, although such protected derivatives of compounds of this disclosure may not possess pharmacological activity as such, they may be administered to a mammal and thereafter metabolized in the body to form compounds of the disclosure which are pharmacologically active. Such derivatives may therefore be described as “prodrugs.” All prodrugs of compounds of this disclosure are included within the scope of the disclosure.

Furthermore, all compounds of this disclosure which exist in free base or acid form can be converted to their pharmaceutically acceptable salts by treatment with the appropriate inorganic or organic base or acid by methods known to one skilled in the art. Salts of the compounds of the disclosure can be converted to their free base or acid form by standard techniques.

The following Reaction Scheme illustrates methods to make compounds of this disclosure, i.e., compounds of Formula (I):

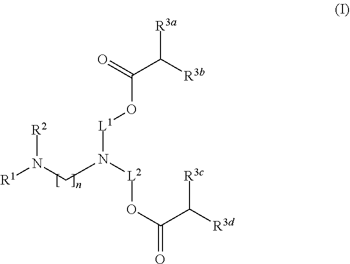

wherein R¹, R², R^{3a}, R^{3b}, R^{3c}, R^{3d}, L¹, L², and n are as defined herein. It is understood that one skilled in the art may be able to make these compounds by similar methods or by combining other methods known to one skilled in the art. It is also understood that one skilled in the art would be able to make, in a similar manner as described below, other compounds of Formula (I) not specifically illustrated below by using the appropriate starting components and modifying the parameters of the synthesis as needed. In general, starting components may be obtained from sources such as Sigma Aldrich, Lancaster Synthesis, Inc., Maybridge, Matrix Scientific, TCI, and Fluorochem USA, etc. or synthesized according to sources known to those skilled in the art (see, for example, Advanced Organic Chemistry: Reactions, Mechanisms, and Structure, 5^{th} edition (Wiley, December 2000)) or prepared as described in this disclosure.

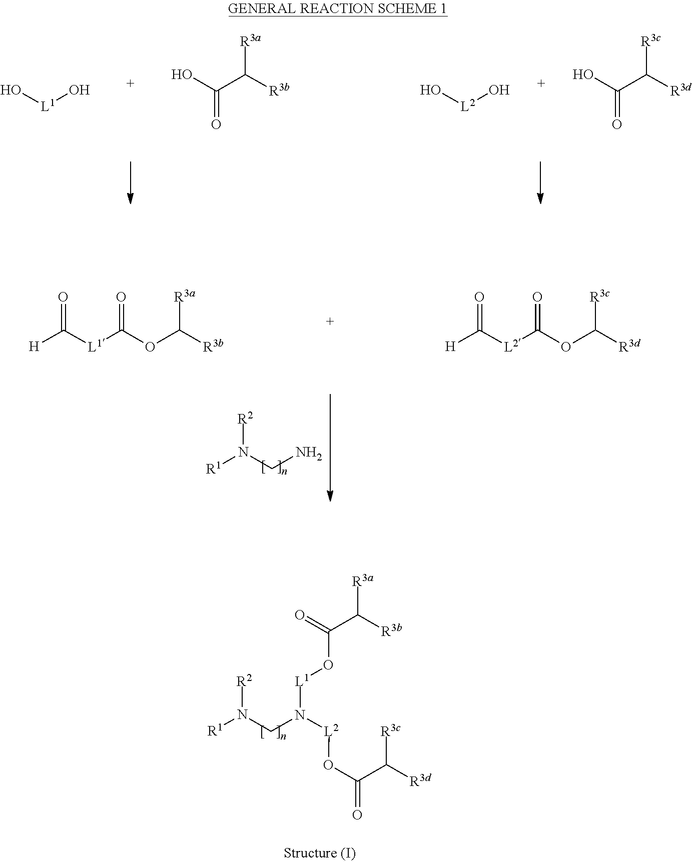

Embodiments of the compound of Formula (I) can be prepared according to General Reaction Scheme 1, wherein R¹, R², R^{3a}, R^{3b}, R^{3c}, R^{3d}, L¹, L², and n are as defined herein (e.g., in 5 claim 1). L^{1′} and L^{2′} are linkers that are converted to L¹ and L² (respectively) when reacted with the N,N-dialkyldiamine as shown.

Referring to General Reaction Scheme 1, starting materials can be purchased from commercial sources or prepared according to methods familiar to one of ordinary skill in the art. The starting materials shown above and DMAP are treated with DCC to give the aldehyde ester compounds as shown. For the next reaction, a mixture of the aldehyde ester compounds are mixed with appropriate reagents (e.g., N,N-dimethylaminoethylamine, acetic acid, and sodium triacetoxyborohydride) and the N,N-dialkyldiamine shown and the reaction mixture allowed to react for a time sufficient to produce compounds of Formula (I) after any necessarily workup and or purification step(s).

### Synthetic Example 1

### Preparation of Compound I-1

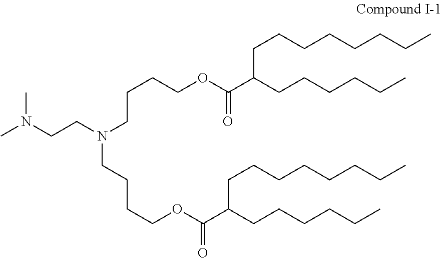 [SMILES: CCCCCCCCC(CCCCCC)C(=O)OCCCCN(CCCCOC(=O)C(CCCCCC)CCCCCCCC)CCN(C)C]

### Step 1: Synthesis of 4-oxobutyl 2-hexyldecanoate

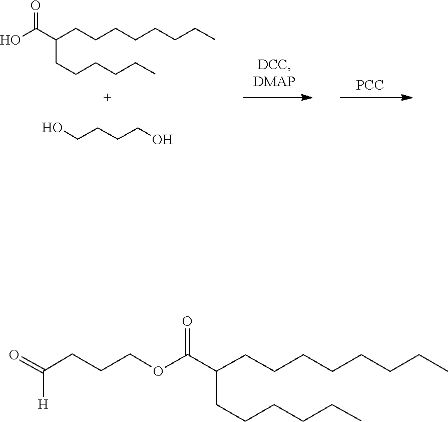 [SMILES: CCCCCCCCC(CCCCCC)C(=O)O ; "PCC" ; OCCCCO ; CCCCCCCCC(CCCCCC)C(=O)OCCCC=O]

A solution of butan-1,2-diol (12.5 g) and 2-hexyldecanoic acid (9.2 g) in dichloromethane (200 mL) and tetrahydrofuran (40 mL) was treated with 4-N,N-dimethylaminopyridine (4.8 g) and N,N′-dicyclohexylcarbodiimide (8.8 g) at room temperature overnight. The solution was filtered, washed with dilute hydrochloric acid, dried over anhydrous magnesium sulfate, filtered, and passed through a silica gel plug. The solvent was removed in vacuo. The residue dissolved in dichloromethane (100 mL) and treated with pyridinium chlorochromate (6 g) for an hour. Diethyl ether (450 mL) was added, and the reaction mixture was filtered through a silica gel bed. The solvent was removed, and the residue was dissolved in hexane (200 mL). The solution was allowed to stand overnight and then filtered through a silica gel bed. Removal of the solvent yielded 11.2 g of desired product.

### Step 2: Synthesis of ((2-(dimethylamino)ethyl)azanediyl)bis(butane-4,1-diyl)bis(2-hexyldecanoate) (Compound I-1)

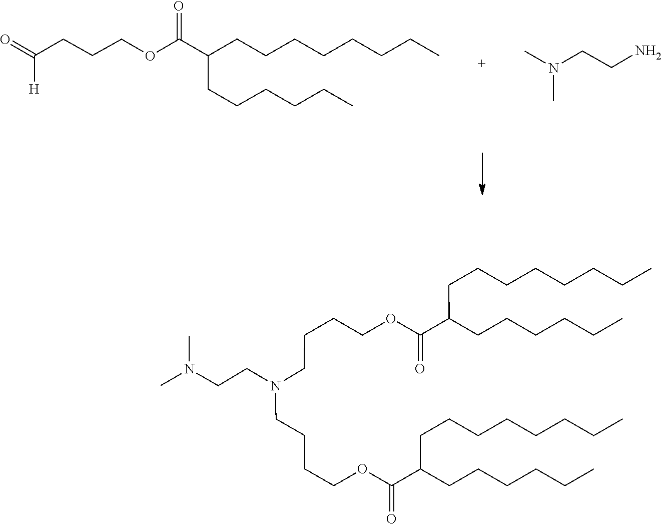

A solution of 4-oxobutyl 2-hexyldecanoate (3 g) in dichloromethane (30 mL) was treated with N,N-dimethylaminoethylamine (0.27 g), acetic acid (0.44 g) and sodium triacetoxyborohydride (1.52 g) for two hours. The reaction mixture was washed with dilute sodium hydroxide solution, dried over anhydrous magnesium sulfate, filtered, and the solvent removed. The crude product was purified by passing down silica gel columns using 2% acetic acid/dichloromethane followed by a methanol/dichloromethane gradient, yielding the product as a colorless oil (0.28 g).

¹H NMR (400 MHz, CDCl₃ at 7.27 ppm) 6: 4.06 (t, 4H, J=6.5 Hz), 2.53 (dd, 2H, J=5.9 Hz, J=8.8 Hz), 2.45 (t, 4H, J=7.4 Hz), 2.26-2.37 (m, 4H), 2.23 (s, 6H), 1.36-1.66 (m, 16H), 1.18-1.32 (m, 40H), 0.86 (t, 6H, J=6.6 Hz).

### Formulation Example 1

### Lipid Nanoparticle Formation

A cationic lipid, DSPC, cholesterol, and pegylated lipid(s) are solubilized in ethanol at desirable molar percentages (e.g., 50:10:38.5:1.5 or 47.5:10:40.7:1.8). Lipid nanoparticles (LNP) are prepared at a total lipid to mRNA weight ratio of approximately 10:1 to 30:1. The mRNA is diluted to 0.2 mg/mL in 10 to 50 mM citrate buffer, pH 4 or 10 to 25 mM acetate buffer, pH 4. Syringe pumps are used to mix the ethanolic lipid solution with the mRNA aqueous solution at a ratio of about 1:5 to 1:3 (vol/vol) with total flow rates above 15 mL/min. The ethanol is then removed, and the external buffer replaced with PBS by dialysis. Finally, the lipid nanoparticles are filtered through a 0.2 m pore sterile filter. Lipid nanoparticle particle size is determined using quasi-elastic light scattering via a Nicomp 370 submicron particle sizer (Santa Barbara, CA). Alternatively, particle size can also be as determined by quasi-elastic light scattering using a Malvern Zetasizer Nano ZS (Malvern, UK).

The various embodiments described above can be combined to provide further embodiments. All of the U.S. patents, U.S. patent application publications, U.S. patent applications, foreign patents, foreign patent applications and non-patent publications referred to in this specification are incorporated herein by reference, in their entirety. Aspects of the embodiments can be modified, if necessary to employ concepts of the various patents, applications, and publications to provide yet further embodiments.

These and other changes can be made to the embodiments considering the above-detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

1. A compound having the following Formula (I): 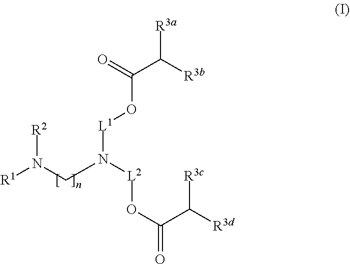
or a pharmaceutically acceptable salt, tautomer, or stereoisomer thereof, wherein:
R¹ and R² are each independently optionally substituted C₁-C₆ alkyl;
R^{3a}, R^{3b}, R^{3c}, and R^{3d} are each independently optionally substituted C₄-C₁₂ alkyl;
L¹ and L² are each independently C₃-C₅ alkylene;
n is 2, 3, 4, or 5.

2. The compound of claim 1, wherein R¹ is unsubstituted or substituted with —OH, —NH₂, alkoxy, or halo.

3. (canceled)

4. The compound of claim 1, wherein R² is unsubstituted or substituted with —OH, —NH₂, alkoxy, or halo.

5. (canceled)

6. The compound of claim 1, wherein R¹ is methyl, ethyl, isopropyl, or n-propyl.

7. (canceled)

8. The compound of claim 1, wherein R² is methyl, ethyl, isopropyl, or n-propyl.

9. (canceled)

10. The compound of claim 1, wherein R¹ and R² are both methyl.

11. The compound of claim 1, wherein L¹ is unsubstituted.

12. The compound of claim 1, wherein L² is unsubstituted.

13-14. (canceled)

15. The compound of claim 1, wherein L¹ and L² are both C₄ alkylene.

16. The compound of claim 1, wherein R^{3a} is C₆-C₁₀ alkyl.

17. (canceled)

18. The compound of claim 1, wherein R^{3b} is C₄-C₈ alkyl.

19. (canceled)

20. The compound of claim 1, wherein R^{3c} is C₆-C₁₀ alkyl.

21. (canceled)

22. The compound of claim 1, wherein R^{3d} is C₄-C₈ alkyl.

23. (canceled)

24. The compound of claim 1, wherein R^{3a} is C₁₀-C₁₂ alkyl.

25. (canceled)

26. The compound of claim 1, wherein R^{3b} is C₁₀-C₁₂ alkyl.

27. (canceled)

28. The compound of claim 1, wherein R^{3c} is C₁₀-C₁₂ alkyl.

29. (canceled)

30. The compound of claim 1, wherein R^{3d} is C₁₀-C₁₂ alkyl.

31-33. (canceled)

34. The compound of claim 1, wherein R^{3a}, R^{3b}, R^{3c}, and R^{3d} are unsubstituted.

35. The compound of claim 1, wherein R^{3a} and R^{3c} are C₈ alkyl and R^{3b} and R^{3d} are C₆ alkyl.

36. The compound of claim 1, wherein R^{3a} and R^{3c} are C₁₀ alkyl and R^{3b} and R^{3d} are C₁₂ alkyl.

37. The compound of claim 1, wherein the compound has one of the following structures:

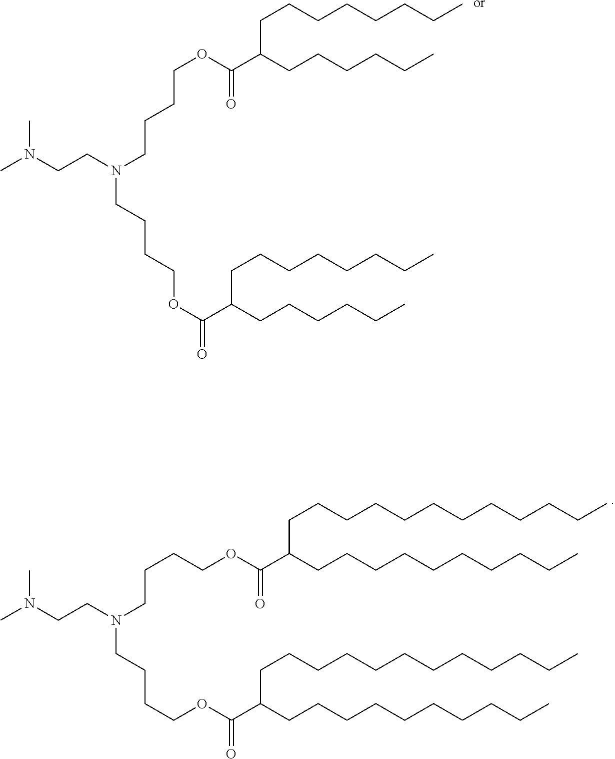

38. A lipid nanoparticle comprising the compound of claim 1 and a therapeutic agent.

39. A composition comprising the compound of claim 1, and a therapeutic agent.

40-55. (canceled)

56. A pharmaceutical composition comprising the lipid nanoparticle of claim 38 and a pharmaceutically acceptable diluent or excipient.

57. A method for administering a therapeutic agent to a patient in need thereof, the method comprising preparing or providing the lipid nanoparticle of claim 38 and administering the composition to the patient.
